(19) United States
(12) Patent Application Publication
(10) Pub. No.: US 2005/0159815 A1
(43) Pub. Date: Jul. 21, 2005

(54) Intervertebral spacer

(75) Inventors: Mikio Kamimura, Nagano-ken (JP); Yukio Taniguchi, Saitama-ken (JP)

Correspondence Address: GREENBLUM & BERNSTEIN, P.L.C., 1950 ROLAND CLARKE PLACE, RESTON, VA, 20191, US

(73) Assignees: PENTAX Corporation, Tokyo (JP); PENTAX Sales Co. Ltd., Tokyo (JP); Mikio KAMIMURA, Nagano (JP)

(21) Appl. No.: 10/845,226

(22) Filed: May 14, 2004

(30) Foreign Application Priority Data
May 14, 2003 (JP) 2003-135509

(51) Int. Cl. A61F 2/44

(52) U.S. Cl. 623/17.11

(57) ABSTRACT

An intervertebral spacer is to be inserted between two vertebral bodies. The intervertebral spacer including a pair of block bodies. Each of the block bodies has an abdominal side portion to be directed to an abdominal side of a patient when inserted in the vertebral bodies and a back side portion to be directed to a back side of the patient when inserted in the vertebral bodies. Each block body is configured such that a width of the abdominal side portion is greater than a width of the back side portion.

### BACKGROUND OF THE INVENTION

The present invention relates to an intervertebral spacer to be inserted between two vertebral bodies.

Spinal canal stenosis typically exhibits, as pathologic condition thereof, a degeneration of an intervertebral disk, a degenerative intervertebral joint disease, a secondary deformation of the vertebral body, a spinal deformation and disturbances of cauda equina and nerve root disorder accompanying these diseases. Conventionally, for a treatment of the spinal canal stenosis, a vertebral body fusion operation is typically done. The vertebral body fusion operation is performed by removing the degenerated intervertebral disk from the intervertebral area, and filling autogenous bone in the intervertebral area to adhering two vertebral bodies then.

However, since the autogenous bone is absorbed by the adjacent vertebral bodies before the synostosis has been completed, the vertebral bodies may possibly be unstably supported. Furthermore, due to a limited volume of collectable autogenous bones, it is sometimes difficult to obtain a sufficient volume of the autogenous bone.

Therefore, an alternative method is generally employed, in which an intervertebral spacer is used as substitute material for the autogenous bone, and the intervertebral spacer is inserted in the intervertebral area together with the autogenous bone. According to this method, the vertebral bodies can be stably supported.

When the above-described method is employed, the intervertebral spacer is required to stably support the adjacent vertebral bodies and required to be fused with the vertebral bodies easily. In view of such requirements, various intervertebral spacers made of a variety of materials and having a variety of shapes have been developed. An example of the intervertebral spacer is shown in Japanese Patent Provisional Publication No. HEI09-122160. The intervertebral space disclosed in this publication is kidney-shaped and formed with a vertical through hole, and is configured to be used together with the autogenous bone. The intervertebral spacer as disclosed is adapted to be inserted in a front (toward the abdomen of a patient) portion of the intervertebral area. The autogenous bone is filled in a back (toward the back of the patient) portion of the intervertebral area as well as in the through hole thereof.

The above-described conventional intervertebral spacer is configured such that a portion that supports the vertebral bodies is very small, therefore, should be made of high strength material. In other words, a range of choice for the material of the spacer is extremely limited. Furthermore, according to the above-described spacer, the vertebral bodies can be stably supported in a side-to-side direction, but, it is difficult to support the vertebral bodies in a front-to-back direction stably. Moreover, the above-described intervertebral spacer may not be used appropriately due to an inappropriate size of the spacer and/or depending on cases.

### SUMMARY OF THE INVENTION

The present invention is advantageous in that an improved intervertebral spacer, which enables a distance between vertebral bodies to be held appropriately. Further, the present invention is advantageous in that the intervertebral spacer that allows an appropriate treatment regardless of the cases.

According to an aspect of the invention, there is provided an intervertebral spacer to be inserted between two vertebral bodies, including a pair of block bodies, each of the block bodies having an abdominal side portion to be directed to an abdominal side of a patient when inserted in the vertebral bodies and a back side portion to be directed to a back side of the patient when inserted in the vertebral bodies, each of the block bodies being configured such that a width of the abdominal side portion is greater than a width of the back side portion.

Optionally, each of the block bodies may be configured such that one side surface is a planar surface having substantially no step, and the other side surface may be formed with at least one step.

In a particular case, there is only one step on the other side surface of each of the block bodies at a position where the abdominal side portion and the back side portion are connected. The width of the abdominal side portion and the width of the back side portion may be substantially constant, respectively.

In one embodiment, a ratio W₂/W₁ of the width W₂ of the back side portion to the width W₁ of the abdominal side portion is within a range of 0.4 to 0.8.

Further optionally, each of the block bodies may be configured such that one side surface is convex surface having substantially no step, and the other side surface may be formed with at least one step.

Furthermore, each of two opposing side surfaces of each of the block bodies may be formed with at least one step.

Still optionally, a ratio L₂/L₁ of a length L₂ of the back side portion to a length L₁ of each of the block bodies may be within a range of 0.4 to 0.8.

Further, the two block bodies may be arranged when inserted in the intervertebral area with the side surfaces being faced each other. In a particular case, the two block bodies have symmetrical shapes.

Optionally, the pair of the block bodies are inserted between the two vertebral bodies such that the one side surfaces thereof face each other.

Alternatively, the pair of the block bodies are inserted between the two vertebral bodies such that the other side surfaces thereof face each other.

Further optionally, each of the block bodies may include a portion whose height gradually decreases from the abdominal side end of the portion toward the back side end of the portion.

Furthermore optionally, at least one of a top surface and bottom surface of each of the block bodies may be a convex surface.

Still optionally, each of the block bodies may be configured such that corners thereof have rounded shapes.

Further, a pair of grooves are formed on opposing side surfaces at opposing positions, the grooves being formed to extend in the vertical direction in a condition where said block body is inserted between the vertebral bodies.

Optionally, a porosity of each of the block bodies is within a range of 0% to 60%.

In a particular case, primary material of the block bodies is ceramic material. Optionally, the ceramic material includes calcium phosphate compound. Further optionally, the calcium phosphate compound has a Ca/P ratio within a range of 1.0 to 2.0. Furthermore, the calcium phosphate compound comprises hydroxyapatite.

In an embodiment, a filler is filled in a space defined by the pair of block bodies and the vertebral bodies. In a particular case, the filler is a transplantation bone.

Optionally, each block body has a position having a maximum height between the abdominal side end and the back side end, the height of the each block body gradually decreasing from the position having the maximum height toward the abdominal side end and toward the back side end.

According to another aspect of the invention, there is provided an intervertebral spacer to be inserted between two vertebral bodies, comprising a pair of block bodies, each block body having at least one step formed on at least one of side surfaces thereof, each of the block bodies being divided into a first portion and a second portion by the step.

Optionally, a width of the first portion may be greater than a width of the second portion.

According to a further aspect of the invention, there is provided an intervertebral spacer system having a plurality of intervertebral spacers each of which is to be inserted between two vertebral bodies, each of the intervertebral spacers including a pair of block bodies having a predetermined length, different block bodies of different intervertebral spacers having different lengths, each of the block bodies having an abdominal side portion to be directed to an abdominal side of a patient when inserted between the vertebral bodies and a back side portion to be directed to a back side of the patient, each of the block bodies being configured such that a width of the abdominal side portion is greater than a width of the back side portion.

### BRIEF DESCRIPTION OF THE ACCOMPANYING DRAWINGS

FIGS. 1A, 1B and 1C are plan view, front view and side view of a block body constituting an intervertebral spacer, according to an embodiment of the present invention;

FIGS. 2-5 show the intervertebral spacer in use, according to the embodiment of the present invention;

FIG. 6 shows an alternative intervertebral spacer inserted in the intervertebral area, according to a second embodiment of the invention; and

FIG. 7 shows a block body for an intervertebral spacer according to a third embodiment of the invention.

### DESCRIPTION OF THE EMBODIMENTS

Hereinafter, referring to the accompanying drawings, intervertebral spacers according to first through third embodiments of the present invention will be described in detail.

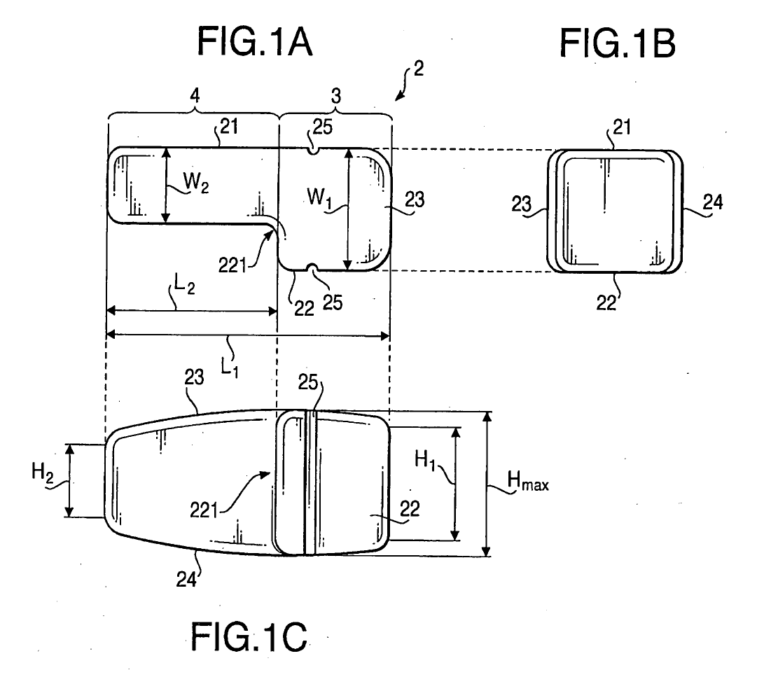

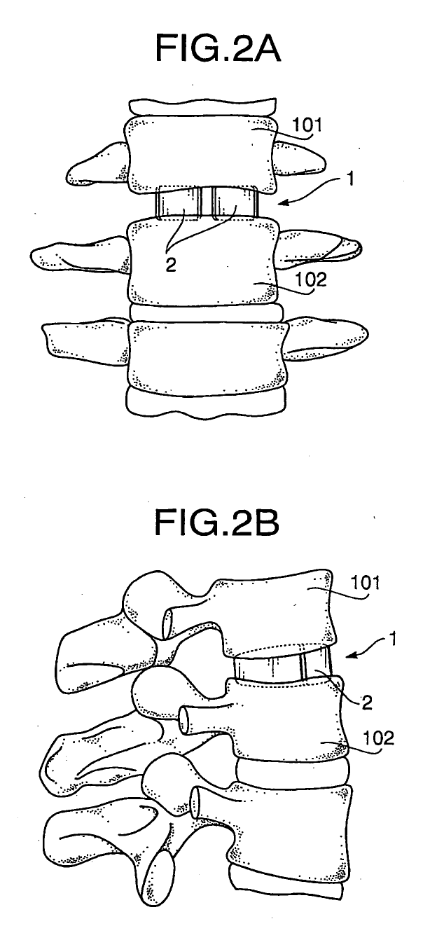

FIGS. 1A through 1C show a trihedral drawing of a block body for an intervertebral spacer 1 according to a first embodiment of the invention. FIG. 1A is a plan view, FIG. 1B is a front view, and FIG. 1C is a side view. FIGS. 2A, 2B, and 3-5 show various states of the intervertebral spacer 1 (hereinafter, occasionally referred to simply as spacer) in use.

It is noted that, directions (sides) referred to in the following description are defined as follows, and portions of the intervertebral spacer and block body are indicated with reference to the directions as defined when the intervertebral spacer (block body) is inserted between vertebral bodies.

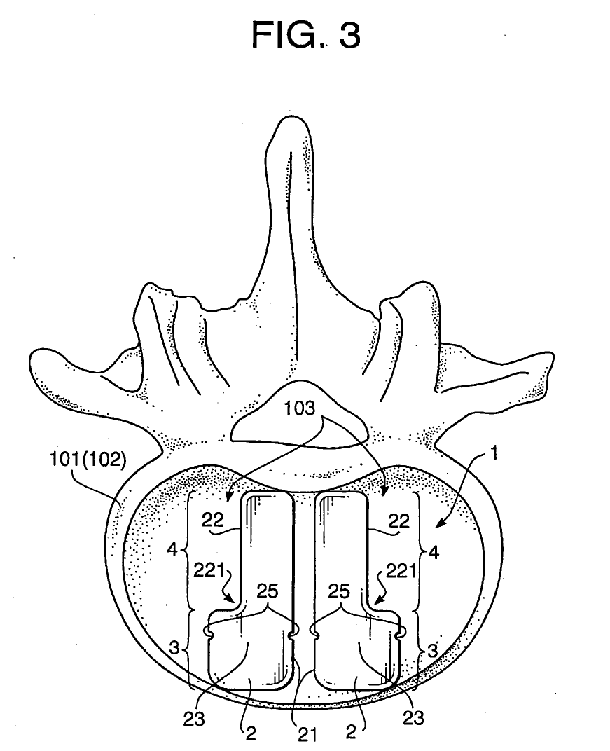

A “front” side is defined as a side, with respect to the vertebral bodies, toward the abdomen of a patient (i.e., right sides of FIGS. 1A, 1C and 2B, front side in a direction perpendicular to a plane of FIG. 2A, and lower sides of FIGS. 3-5). A “back” side is defined as a side, with respect to the vertebral bodies, toward the back of the patient (i.e., left sides of the FIGS. 1A, 1C and 2B, back side in a direction perpendicular to a plane of FIG. 2A, and upper sides of FIGS. 3-5). A “top” side is defined as a side, with respect to the vertebral bodies, toward the head of the patient (i.e., upper sides of FIG. 1C and 2, front sides in the direction perpendicular to a plane of each of FIGS. 1A and 3-5, left-hand side of FIG. 1C). A “bottom” side is defined as a side toward the legs of the patient (bottom sides of FIGS. 1C and 2, back sides through FIGS. 3-5, and right side of FIG. 1C) is defined as “bottom”.

As shown in FIG. 2, the intervertebral spacer 1 is adapted to be inserted between two vertebral bodies (i.e., inserted intervertebrally) 101 and 102 after an intervertebral disk therebetween is removed. The distance between the vertebral bodies 101 and 102 (the distance will be referred to as a “clearance” hereinafter) is kept (held) appropriately when the intervertebral spacer 1 is inserted intervertebrally (this state will be referred to as an “inserted state”).

As shown in FIGS. 2A and 3-5, the intervertebral spacer 1 includes a pair of block bodies 2 and 2. The block bodies are substantially symmetric in shape and structure.

Since each block bodies 2 and 2 has substantially the same structure (although symmetric with each other), only one of the block bodies 2 and 2 will be described in detail and the description on the other block body 2 is omitted.

As shown in FIG. 1A, the block body 2 has a substantially planar side surface 21 and a stepped side surface 22. The side surface 21 is configured to have substantially no steps. The stepped side surface 22 is configured to have a step 221 thereon. As indicated in FIG. 1A, a width W₁ of the block body 2 on its back side is larger than a width W₂ thereof on its front side.

At the step 221, the block body 2 is divided into two portions. One portion on the front side of the block body 2 will be referred to as a front portion 3, and the other portion will be referred to as a rear portion 4. The front portion 3 has a substantially constant width in its longitudinal direction (front-to-back direction), and the rear portion 4 also has a substantially constant width in its longitudinal direction. With this structure, the block body 2 is substantially L-shaped viewed from top or bottom side thereof.

With the shape described above, the spacer 1 can support the vertebral bodies 101 and 102 stably both in the side-to-side direction and front-to-back direction. In particular, the front portion 3 of the block body 2 is configured to have a relatively large strength so as to endure a large compressive force applied thereto from the vertebral bodies 101 and 102. Accordingly, the block body 2 has an excellent durability. As a result, it is ensured that the spacer 1 is capable of maintaining the repositioning function for a long period.

Additionally, when the spacer 1 is inserted, a space occupied by the back portion 4 of each block bodies 2 can be made small on the back side of the intervertebral area between the vertebral bodies 101 and 102. Therefore, a sufficient space 103 to be filled with a filler can be obtained, as shown in FIGS. 3-5. At least a part of the space 103 is defined by the spaces on the inner side of the step 221 of the block bodies 2 and 2. By filling a transplantation bone (preferably an autogenous bone) as the filler, the vertebral bodies 101 and 102 can be synostosis is caused safely and rapidly without fail.

In the following description, the autogenous bone is used as the filler. It should be noted, however, that the invention need not be limited to this configuration, and any other suitable material can be used as the filler.

According to the embodiment, the ratio W₂/W₁ is preferably in a range of 0.4 to 0.8, and more preferably, the ratio is in a range of 0.5 to 0.7. If the ratio W₂/W₁ is below the lower limit of the range, the block body 2 tends to rotate about a longitudinal axis thereof. Accordingly, it may become difficult to insert the block body 2 in the intervertebral area without the rotation of the block body 2, and thereby it becomes difficult to set the block body 2 properly. When the ratio W₂/W₃ exceeds the upper limit of the range, it may be impossible to obtain a sufficient space in which the autogenous bone is filled depending on the size of the vertebral bodies 101 and 102.

Specifically, the width W₁ of the front portion 3 is preferably in a range of 7 mm to 13 mm, and more preferably, W₁ is in a range of 9 mm to 13 mm. The width W₂ Of the rear portion 4 is preferably in a range of 3 mm to 9 mm, and more preferably, W₂ is in a range of 5 mm to 7 mm, with satisfying the above-described ratio.

When the length of the block body 2, along the front-to-back direction, is defined as L₁, and the length from the rear end of the block body 2 to the step 221 (i.e., the length of the rear portion 4), is defined as L₂, a ratio L₂/L₁ is preferably in a range of 0.4 to 0.8, and more preferably, the ratio is in a range of 0.5 to 0.7. When the ratio L₂/L₁ is in the range, a sufficient space for filling the autogenous bone can be obtained on the back side portion of the intervertebral area when the spacer 1 is inserted.

Specifically, the length L₁ of the block body 2 is determined based on the average length of the vertebral body in the front-to-back direction. The length L₁ (exemplary but not limiting value) is preferably in a range of 15 mm to 30 mm, and more preferably in a range of 22.5 mm to 27.5 mm. The length L₂ (exemplary but not limiting value) is preferably in a range of 10 mm to 20 mm, and more preferably, in a range of 2.5 mm to 17.5 mm, with satisfying the above-described ratio.

The top surface 23 and the bottom surface 24 of the block body 2 are inclined to approach from an intermediate position in the longitudinal direction (front-to-back direction) to the back side end. Accordingly, the height (i.e., the thickness) of the rear portion 4 gradually decreases toward the back end of the rear portion 4.

The above-described block body 2 is, especially, suitable for application to a portion in which the distance between the two vertebral bodies 101 and 102 is larger at the back side than at the front side. When the block body 2 configured as above is used for such a portion, the two vertebral bodies 101 and 102 can be held more stably.

As shown in FIG. 1C, each of the top and bottom surfaces 23 and 24 is configured to a curved convex surface. Since the vertebral bodies generally have curved concave top and bottom surfaces, the curved convex surfaces 23 and 24 of the block body 2 well fit to the vertebral bodies. By use of such a block body 2, it is ensured that a displacement of the block bodies 2 and 2 after they are fixed between the vertebral bodies can be prevented.

Each radius of curvature of the top and bottom surfaces 23 and 24 is configured based on the average shapes of the top and bottom surfaces of the vertebral bodies between which the spacer is inserted. Preferably, the radius of curvature is, for example but not limiting, in a range of 50 mm to 70 mm, and more preferably, in a range of 55 mm to 65 mm.

As shown in FIG. 1C, the block body 2 is the highest (Hₘₐₓ) at a position near the step 221, and the height gradually decreases toward the front and back end. The height (H₁) at the front end is larger than the height (H₂) at the back end.

The maximum height Hₘₐₓ of the block body is preferably within a range of 8 mm to 15 mm, and more preferably, within a range of 11 mm to 13 mm. The height H₁ at the front end of the block body 2 is preferably within a range of 7.5 mm to 13.5 mm, and more preferably, within a range of 9.5 mm to 11.5 mm, maintaining the relation of Hₘₐₓ>H₁. The height H₂ of the block body 2 at the back end is preferably within a range of 4.5 mm to 10.5 mm, and more preferably, within a range of 6.5 mm to 8.5 mm, maintaining the relation of H₁>H₂.

Every corners of the block body 2 is formed to have a rounded shape, and the organic tissues are protected from being damaged by the corners when each block body is inserted in the intervertebral area.

Two grooves 25 and 25 extending in the top-to-bottom direction are formed on the two side surfaces 21 and 22 of the block body 2 (on the side surfaces of the front portion 3), respectively The grooves 25 and 25 are located at substantially opposing positions. By positioning tips of a grasping device such as a pair of tweezers on the grooves 25 and 25, the block body 2 can be grasped stably. With the grooves 25 and 25, slipping and falling-off of the block body 2 from the grasping device, when the block body 2 is inserted in or removed from the intervertebral area, can be prevented. That is, the block body 2 can be safely and rapidly inserted in or removed from the intervertebral area as the grooves 25 and 25 prevent slipping.

It should be noted that the positions of the grooves 25 and 25 are not limited to the positions indicated in the figures. The grooves 25 can be determined at arbitrarily positions in the longitudinal direction of the block body 2. Further, the number of the grooves 25 may be more than two.

The block body 2 is preferably made of material which primarily includes ceramic, while it can be made of metallic material such as titanium. The ceramic material is preferable since the material can easily formed into a desired shape using a cutting apparatus such as a lathe and a drilling machine. Furthermore the shape of the block body 2 can be easily modified when a surgical operation is performed. That is, the size of the block bodies can be finely adjusted in accordance with operational cases. For example, the shape and dimension of each block body 2 may be adjusted to be suitable for the shapes and sizes of the vertebral bodies 101 and 102, and/or the degree of the curvature of the vertebral bodies.

Some bioceramics material, such as alumina, zirconia, or calcium phosphate compound, is preferably used as material of the block body 2. In particular, calcium phosphate compound is especially preferable because of its excellent biocompatibility.

Several types of the calcium phosphate compounds, such as apatite material including hydroxyapatite, fluorapatite and carbonate apatite, dicalcium phosphate, tricalcium phosphate, or octcalcium phosphate are available. The block body 2 is preferably made of one of these types of the calcium phosphate compound, or made of mixture of two or more types of the calcium phosphate compounds. Preferably, a calcium phosphate compound having a Ca/P ratio of 1.0 to 2.0 is used as the material of the block body 2.

More preferably, among the above-described calcium phosphate compounds, hydroxyapatite is used as the material of the block body 2. The hydroxyapatite has very excellent biocompatibility since the material has a composition, structure and physical property substantially the same as those of the principal inorganic component of the bone.

For the material of the block body 2, the hydroxyapatite particles are preferably calcined at a temperature of 500° C. to 1000° C. The calcined hydroxyapatite particles exhibit relatively low activity, therefore, inhomogeneous burning occurred by a rapid sintering can be prevented, thereby a sintered body having a homogeneous strength can be obtained.

The block body 2 is preferably configured to have a porosity of 60% or lower, and more preferably, the porosity is in a range of 5% to 30%. The block body 2 has a sufficient strength and can improve the remodeling of the bone by osteoconduction.

It is noted that the block body 2 can be made of composite material including the ceramic material and metallic material which has less harmfulness to the living body. Such metallic material includes, for example, titanium, titanium alloy, stainless steel, Co—Cr alloy and Ni—Ti alloy.

In this embodiment, two block bodies 2 and 2, having symmetric shapes, are arranged in the left-to-right direction and inserted in the intervertebral area.

Since the spacer 1 consists of a pair of block bodies 2 and 2, as described above, various shapes of the spacer 1 can be configured by changing the position of each block body 2.

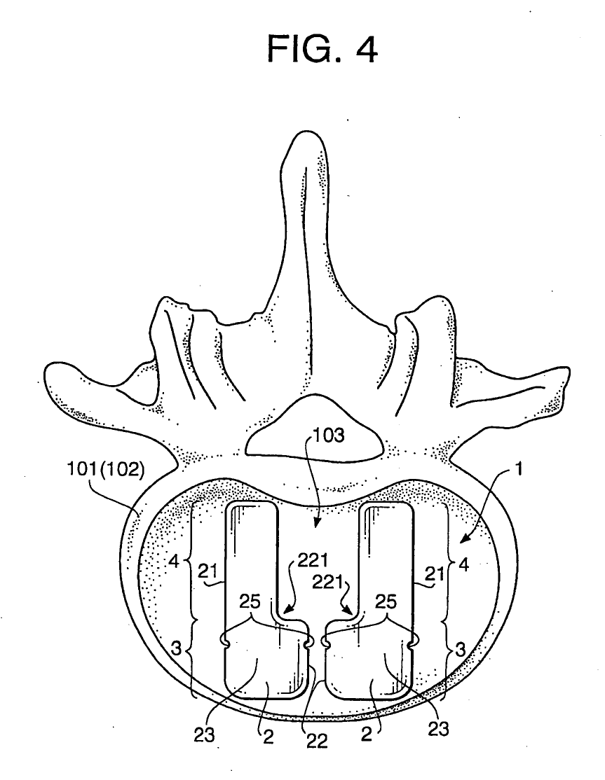

One example of the arrangement of the block bodies 2 and 2 is shown in FIG. 3. In FIG. 3, the side surfaces 21 of block bodies 2 face each other so that the top view of the spacer 1 is T-shaped. Another exemplary arrangement of the block bodies 2 and 2 is shown in FIG. 4. In FIG. 4, the side surfaces 22 of the block bodies 2 face each other so that the top view of the spacer 1 is U-shaped.

When the block bodies 2 and 2 are arranged as shown in FIG. 3, relatively large spaces 103 are defined, by each block body 2 and the vertebral bodies 101 and 102, at left side and right side portions of the intervertebral area. Accordingly, relatively large volume of the autogenous bone can be filled in the intervertebral area.

The volume of the space 103 can be adjusted by modifying the height of the step 221 and/or the length L₁ of each block body 2.

If the length L₁ is relatively small, for example, the volume of the space 103 is relatively large. As a result, large volume of the autogenous bone can be filled in the intervertebral area, and the synostosis of the vertebral bodies 101 and 102 with the autogenous bone is caused earlier. Further, in this case, on a back side area of the intervertebral area with respect to the spacer 1 (each block body 2) is an area in which only the autogenous bone is filled. Therefore, since an X-ray transmission is not prevented by the block bodies 2 in this area, it become easy to diagnose the progress of the synostosis by use of the X-ray photography.

Further, when the block bodies 2 are arrange as shown in FIG. 3, the spacer 1 supports the vertebral bodies 101 and 102 at the central portions thereof. Therefore, the pressure provided by the spacer 1 can be adjusted using pedicle screws. By adjusting the pressure, kyphosis is created and the scoliosis can be corrected easily. Accordingly, the arrangement of the block bodies 2 shown in FIG. 3 is effective for treating a degenerative scoliosis which requires a formation of the kyphosis and the correction of the scoliosis. It should be noted that, for this purpose, the block bodies 2 having relatively small lengths L₁ are especially effective.

When the block bodies are arranged as shown in FIG. 4, each block body 2 can support the whole vertebral bodies 101 and 102 stably. Further, since the space 103 is formed between the block bodies 2 and 2, it is ensured that the autogenous bone can be filled in at a predetermined position.

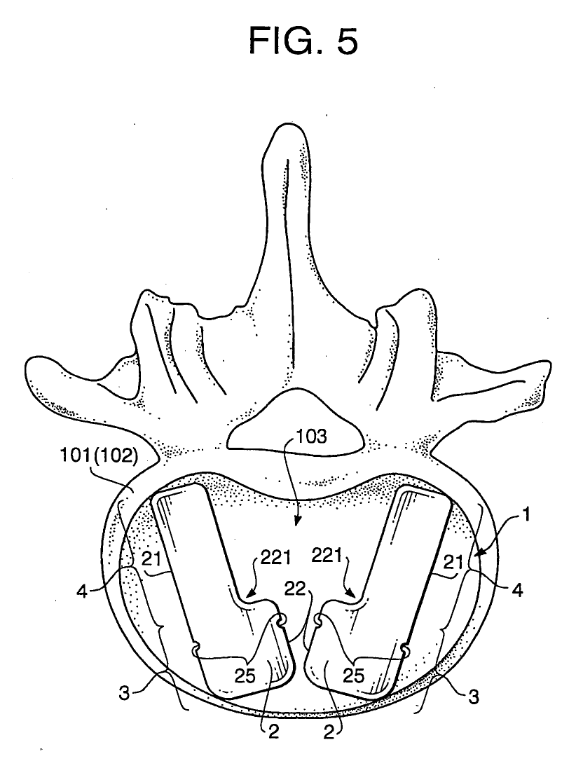

It should be noted that the arrangement of the block bodies 2 and 2 is not limited to those shown in FIGS. 3 and 4. FIG. 5 shows an another example of the arrangement of the block bodies 2 and 2. In the example shown in FIG. 5, the block bodies 2 and 2 are arranged such that a distance between the back ends of the block bodies 2 and 2 is larger than distance between the front ends thereof so that the top view of the spacer 1 is V-shaped. This arrangement is suitable for fixing the spacer 1 to a patient having relatively small vertebral bodies 101 and 102.

As described above in detail, according to the present invention, various treatments corresponding to various symptoms of the vertebral bodies can be appropriately performed by suitably arranging the block bodies 2 and 2.

Incidentally, when a manipulation of inserting the spacer 1 into the vertebral bodies 101 and 102, two or more types of the block bodies 2 and 2 having different lengths L₁ may be prepared and a suitable type of block bodies 2 and 2 may be selected therefrom. In such a case, more various cases can be appropriately treated.

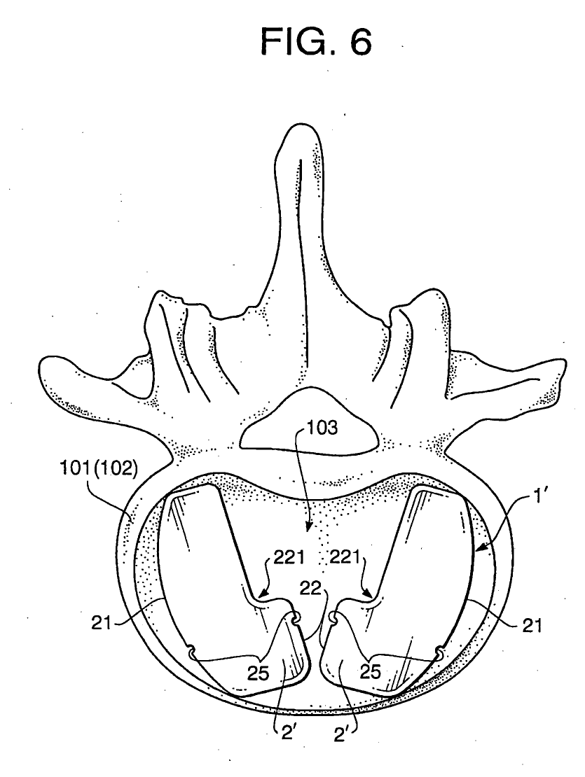

Hereinafter, a intervertebral spacer according to a second embodiment of the present invention will be described. FIG. 6 shows an intervertebral spacer 1′ inserted in the intervertebral area, according to the second embodiment. In the following description, only different portions with respect to the first embodiment will be provided.

In the second embodiment, a shape of the block body 2′ is similar to that of the block body 2 according to the first embodiment except that the block body 2′ has a curved convex side surface 21. Other portion of the block body 2′ are the same as that of the block body 2 of the above-described embodiment.

The convex side surface 21 is preferably formed to have a shape corresponding to the inner curved concave surfaces of the vertebral bodies 101 and 102. With this configuration, the block bodies 2′ and 2′ have relatively large contacting areas that contact the vertebral bodies 101 and 102. By use of the above-configured block bodies 2′ and 2′, the vertebral bodies 101 and 102 can be supported more stably.

The spacer 1′ can be used in the same manner as the spacer 1 according to the first embodiment. That is, the arrangement of the block bodies 2′ and 2′ can be varied depending on the cases as exemplified in FIGS. 3-5.

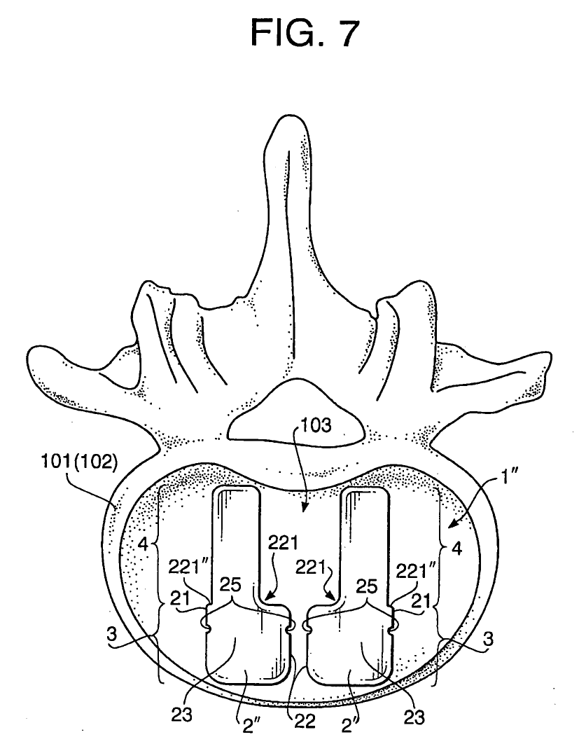

FIG. 7 shows a block body 2″ of the spacer 1″, according to a third embodiment of the present invention.

According to the third embodiment, as shown in FIG. 7, another step 221″ is formed on the side surface 21 as well as the step 221 formed on the side surface 22. The spacer 1″ can also be used in the similar manner as the spacers 1 and 1′.

In the above-described embodiments, the autogenous bone or other transplantation bones are used as a filler filled in the intervertebral area. The filler need not be limited to those described above, and other types of the filler such as a powder made from calcium phosphate compound or other materials can be also used. Optionally, the filler may include materials for improving the bone synostosis, such as BMP (Bone Morphogenetic Protein), TGF (Transforming Growth Factor) and the like.

As described above, according to the present invention, the distance between two vertebral bodies can be maintained appropriately. Furthermore, the appropriate treatment can be performed in accordance with various cases occurred to the vertebral bodies.

Further, according to the embodiments and modifications described above, since the sufficient space for filling in the filler can be obtained in the intervertebral area, the synostosis of the vertebral bodies can be improved by filling a transplantation bone in the space.

Although the present invention has been described with reference to the particular illustrative embodiments, the scope of the invention is not to be restricted by those embodiments but only by the appended claims. It is to be appreciated that those skilled in the art can change or modify the embodiments without departing from the scope of the present invention.

The present disclosure relates to the subject matter contained in Japanese Patent Application No. 2003-135509, filed on May 14, 2003, which is expressly incorporated herein by reference in its entirety.

1. An intervertebral spacer to be inserted between two vertebral bodies, comprising a pair of block bodies, each of the block bodies having an abdominal side portion to be directed to an abdominal side of a patient when inserted in the vertebral bodies and a back side portion to be directed to a back side of the patient when inserted in the vertebral bodies, each of the block bodies being configured such that a width of the abdominal side portion is greater than a width of the back side portion.

2. The intervertebral spacer according to claim 1, wherein each of the block bodies is configured such that one side surface is a planar surface having substantially no step, and the other side surface is formed with at least one step.

3. The intervertebral spacer according to claim 2, wherein there is only one step on the other side surface of each of the block bodies at a position where the abdominal side portion and the back side portion are connected, the width of the abdominal side portion being substantially constant, the width of the back side portion being substantially constant.

4. The intervertebral spacer according to claim 3, wherein a ratio W₂/W₁ of the width W₂ of the back side portion to the width W₁ of the abdominal side portion is within a range of 0.4 to 0.8.

5. The intervertebral spacer according to claim 1, wherein each of the block bodies is configured such that one side surface is convex surface having substantially no step, and the other side surface is formed with at least one step.

6. The intervertebral spacer according to claim 1, wherein each of two opposing side surfaces of each of the block bodies is formed with at least one step.

7. The intervertebral spacer according to claim 1, wherein a ratio L₂/L₃ of a length L₂ of the back side portion to a length L₁ of each of the block bodies is within a range of 0.4 to 0.8.

8. The intervertebral spacer according to claim 2 wherein the two block bodies are arranged when inserted in the intervertebral area with the side surfaces being faced each other.

9. The intervertebral spacer according to claim 8, wherein the two block bodies have symmetrical shapes.

10. The intervertebral spacer according to claim 9, wherein the pair of the block bodies are inserted between the two vertebral bodies such that the one side surfaces thereof face each other.

11. The intervertebral spacer according to claim 9, wherein the pair of the block bodies are inserted between the two vertebral bodies such that the other side surfaces thereof face each other.

12. The intervertebral spacer according to claim 1, wherein each of the block bodies include a portion whose height gradually decreases from the abdominal side end of the portion toward the back side end of the portion.

13. The intervertebral spacer according to claim 1, wherein at least one of a top surface and bottom surface of each of the block bodies is a convex surface.

14. The intervertebral spacer according to claim 1, wherein each of the block bodies is configured such that corners thereof have rounded shapes.

15. The intervertebral spacer according to claim 1, wherein a pair of grooves are formed on opposing side surfaces at opposing positions, the grooves being formed to extend in the vertical direction in a condition where said block body is inserted between the vertebral bodies.

16. The intervertebral spacer according to claim 1, wherein a porosity of each of the block bodies is within a range of 0% to 60%.

17. The intervertebral spacer according to claim 1, wherein primary material of the block bodies is ceramic material.

18. The intervertebral spacer according to claim 17, wherein the ceramic material includes calcium phosphate compound.

19. The intervertebral spacer according to claim 18, wherein the calcium phosphate compound has a Ca/P ratio within a range of 1.0 to 2.0.

20. The intervertebral spacer according to claim 19, wherein the calcium phosphate compound comprises hydroxyapatite.

21. The intervertebral spacer according to claim 1, wherein a filler is filled in a space defined by the pair of block bodies and the vertebral bodies.

22. The intervertebral spacer according to claim 21, wherein the filler is a transplantation bone.

23. The intervertebral spacer according to claim 1, wherein each block body has a position having a maximum height between the abdominal side end and the back side end, the height of the each block body gradually decreasing from the position having the maximum height toward the abdominal side end and toward the back side end.

24. An intervertebral spacer to be inserted between two vertebral bodies, comprising a pair of block bodies, each block body having at least one step formed on at least one of side surfaces thereof, each of the block bodies being divided into a first portion and a second portion by the step.

25. The intervertebral spacer according to claim 24, wherein a width of the first portion is greater than a width of the second portion.

26. An intervertebral spacer system having a plurality of intervertebral spacers each of which is to be inserted between two vertebral bodies, each of the intervertebral spacers comprising a pair of block bodies having a predetermined length, different block bodies of different intervertebral spacers having different lengths, each of the block bodies having an abdominal side portion to be directed to an abdominal side of a patient when inserted between the vertebral bodies and a back side portion to be directed to a back side of the patient, each of the block bodies being configured such that a width of the abdominal side portion is greater than a width of the back side portion.
